(19) United States
(12) Patent Application Publication
(10) Pub. No.: US 2022/0392649 A1
(43) Pub. Date: Dec. 8, 2022

(54) CLINICAL ASSOCIATION EVALUATING APPARATUS AND CLINICAL ASSOCIATION EVALUATING METHOD

(71) Applicant: AESOP Technology Inc., San Francisco, CA (US)

(72) Inventors: Yu-Chuan Li, Taipei City (TW); An Jim Long, Taipei City (TW)

(73) Assignee: AESOP Technology Inc., San Francisco, CA (US)

(21) Appl. No.: 17/412,262

(22) Filed: Aug. 26, 2021

Related U.S. Application Data
(60) Provisional application No. 63/196,186, filed on Jun. 2, 2021.

(51) Int. Cl. G16H 70/60

(52) U.S. Cl. CPC G16H 70/60

(57) ABSTRACT

A clinical association evaluating apparatus and a clinical association evaluating method are provided. In the method, association coefficients between multiple diseases and medicines are determined through an evaluating model. The evaluating model is trained through a machine learning algorithm. The first association between each medicine and the disease with the highest association coefficient is maintained, and the second association between each medicine and the disease without the highest association coefficient is disconnected. The association coefficients between the medicines and the diseases are modified according to the maintained first association and the disconnected second association. Accordingly, the modified association coefficients are adapted for clinical application, and false associations could be removed.

### CROSS-REFERENCE TO RELATED APPLICATION

This application claims the priority benefit of U.S. provisional application Ser. No. 63/196,186, filed on Jun. 2, 2021. The entirety of the above-mentioned patent application is hereby incorporated by reference herein and made a part of specification.

### BACKGROUND

### Technical Field

The disclosure relates to a clinical medical evaluation technology, and in particular to a clinical association evaluating apparatus and a clinical association evaluating method.

### Description of Related Art

From data mining to deep learning, the exploration of associations between clinical variables is an important task of machine learning that is imported into clinical applications. However, a conventional probabilistic model and neural network cannot effectively address common clinical comorbidity, and further create false associations which cause invalid clinical findings.

### SUMMARY

In view of the above, the embodiments of the disclosure provide a clinical association evaluating apparatus and a clinical association evaluating method, to increase the differences between different combinations and exclude false associations accordingly.

A clinical association evaluating method according to an embodiment of the disclosure includes (but not limited to) the followings. Association coefficients between multiple diseases and multiple medicines are determined through an evaluating model; and the evaluating model is trained through a machine learning algorithm. A first association between each of the medicines and one of the diseases with the highest association coefficient is maintained, and a second association between each of the medicines and at least one of the diseases without the highest association coefficient is disconnected. According to the first association that is maintained and the second association that is disconnected, the association coefficients between the diseases and the medicines are modified through the evaluating model.

A clinical association evaluating apparatus according to an embodiment of the disclosure includes (but not limited to) a storage device and a processor. The storage device is used for storing a code. The processor is coupled to the storage device and is configured to load and execute the code to execute the followings. Association coefficients between a plurality of diseases and a plurality of medicines are determined through an evaluating model. A first association between each of the medicines and one of the diseases with the highest association coefficient is maintained, and a second association between each of the medicines and at least one of the diseases without the highest association coefficient is disconnected. According to the first association that is maintained and the second association that is disconnected, the association coefficients between the diseases and the medicines through the evaluating model are modified. The evaluating model is trained through a machine learning algorithm.

Based on the above, the clinical association evaluating apparatus and the clinical association evaluating method according to the embodiments of the disclosure increase the association coefficients of the strong association combination (that is, the combinations with the highest association coefficient between a specific medicine and a specific disease), and reduce the association coefficients of the weak association combination (that is, the combinations without the highest association coefficient between a specific medicine and a specific disease). In this way, the difference between the two combinations is increased, and false associations are removed.

To provide a further understanding of the above features and advantages of the disclosure, embodiments accompanied with drawings are described below in details.

### BRIEF DESCRIPTION OF THE DRAWINGS

FIG. 1 is a block diagram of elements of a clinical association evaluating apparatus according to an embodiment of the disclosure.

FIG. 2 is a flow chart of a clinical association evaluating method according to an embodiment of the disclosure.

### DESCRIPTION OF THE EMBODIMENTS

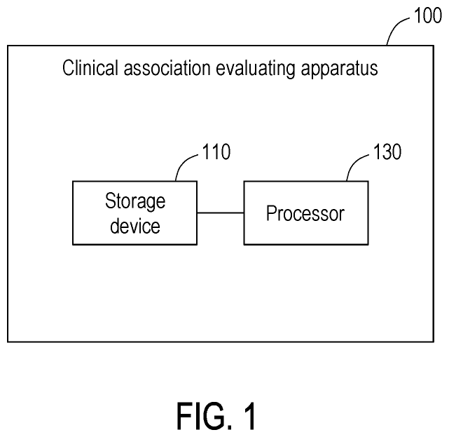

FIG. 1 is a block diagram of elements of a clinical association evaluating apparatus 10 according to an embodiment of the disclosure. Referring to FIG. 1, the clinical association evaluating apparatus 100 includes (but not limited to) a storage device 110 and a processor 130. The clinical association evaluating apparatus 100 may be a desktop computer, a notebook computer, a smart phone, a tablet computer, a server, a medical testing instrument, or other computing devices.

The storage device 110 may be any type of fixed or removable random access memory (RAM), read only memory (ROM), flash memory, hard disk drive (HDD), solid-state drive (SSD) or a similar element. In an embodiment, the storage device 110 is used to record codes, software modules, configurations, data (for example, association variables, association coefficients, models, etc.) or files, and the embodiment will be described in detail later.

The processor 130 is coupled to the storage device 110, and the processor 130 may be a central processing unit (CPU), a graphics processing unit (GPU), or other programmable general-purpose or special-purpose elements such as a microprocessor, a digital signal processor (DSP), a programmable controller, a field programmable gate array (FPGA), an application-specific integrated circuit (ASIC), a neural network accelerator or other similar elements or a combination of the above elements. In an embodiment, the processor 130 is used to execute all or part of the operations of the clinical association evaluating apparatus 100, and may load and execute various codes, software modules, files, and data recorded by the storage device 110.

Hereinafter, each element and module in the clinical association evaluating apparatus 100 will be used to describe the method described in the embodiment of the disclosure. Each process of this method may be adjusted based on different implementation situations, and is not limited to thereto.

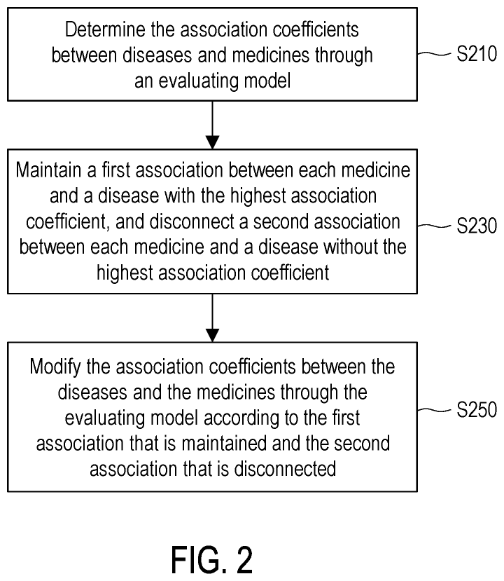

FIG. 2 is a flow chart of a clinical association evaluating method according to an embodiment of the disclosure. Referring to FIG. 2, the processor 130 determines the association coefficients between multiple diseases and multiple medicines through an evaluating model (step S210). Specifically, the evaluating model is trained through a machine learning algorithm. The machine learning algorithm may be a supervised learning algorithm or an unsupervised learning algorithm. The machine learning algorithm may analyze training samples to obtain a pattern from the training samples, so as to predict unknown data through the pattern. The evaluating model is a machine learning model constructed after being trained, and inferences on the data to be evaluated are made based on the evaluating model.

In an embodiment, the evaluating model uses actual prescription medicines and diseases as the training samples. In addition, the association coefficients are related to the degree of association between medicines and diseases. For example, a higher association coefficient indicates a higher degree of association between a medicine and a disease, and may mean that the medicine is included in most of the prescriptions for the disease (but not limited to thereto). Alternatively, a lower association coefficient indicates a lower degree of association between a medicine and a disease, and may mean that the medicine is not included in all the prescriptions for the disease (but not limited to thereto).

For example, in Document 1, “Improved diagnosis medicine association mining to reduce pseudo-associations” accepted in Computer Methods and Programs in Biomedicine in May 2021, a secondary coefficient is used to calculate the association coefficients between diagnosis and medicines and the association coefficients between diagnosis and tests, thereby reducing false associations.

In an embodiment, the evaluating model is a probabilistic model. The probabilistic model is an unsupervised learning algorithm and is an important method of data mining. For example, in Document 2, “An automated technique for identifying associations between medications, laboratory results and problems” on pages 891 to 901 of Biomedical Information Journal, volume 43, issue 6, December 2020, a conviction coefficient is used to calculate the association coefficients between diagnosis and medicines and the association coefficients between diagnosis and tests, and the relationship between the above two pairs of clinical variables (for example, diagnosis and medicines, and diagnosis and tests) may be determined based on the strength of the association coefficients.

As another example, in Document 3, “A Probabilistic Model for Reducing Medicine Errors” published by https://doi.org/10.1371/journal.pone.0082401 in December 2013, a similar model Q coefficient is used to calculate the correlation coefficients between diagnosis and medicines and the correlation coefficients between medicines and medicines, and an appropriateness of prescription (AOP) model is used to evaluate the appropriateness of a prescription. A range of the Q coefficient is defined within [0, ∞]; Q=1 means that there is no association between a disease and a medicine; Q<1 means that a disease and a medicine are negatively correlated; and Q>1 means that a disease and a medicine are positively correlated.

In another embodiment, the evaluating model is a neural network model. For example, a deep neural network (DNN) has a main architecture of a neural network. This deep neural network architecture includes an input layer, a hidden layer, and an output layer. It is to be noted that the deep neural network is formed by a multi-layer neuron structure, and each layer of neurons is configured with an input (for example, an output of a previous layer of neurons) and an output. The neurons in any layer of the hidden layer, through the inner product of an input vector and a weight vector, output a scalar result through a nonlinear transfer function. In the learning stage of the evaluating model, the aforementioned weight vector is trained and determined. Alternatively, while in the inference stage of the evaluating model, the determined weight vector is used to obtain an evaluation result (that is, the output). In this embodiment, the evaluation result of the evaluating model is the association coefficients between input clinical variables. The association coefficients may be probabilities, Q coefficients, or other quantitative values. The clinical variables include medicines, diseases, patient characteristics (for example, gender, age, race, socioeconomic status, or weight), and/or visit categories (for example, clinic or hospital, area of visit such as Taipei, Taiwan or California, USA, emergency hospitalization or clinic service, or the department of the treatment).

For example, Table (1) illustrates the correspondence between diseases, medicines, and association coefficients obtained by the evaluating model:

TABLE 1

| | | Association coefficient |
|---|---|---|
| | | (taking the Q coefficient |
| Disease | Medicine | as an example) |
| Type 2 diabetes mellitus | Metformin | 17.25299 |
| | Captopril | 4.256197 |
| Hypertension | Metformin | 5.134554 |
| | Captopril | 5.442649 |

It is to be noted that false associations might not be excluded from association coefficients derived from an existing probabilistic model or neural network model. For example, although Document 2 discloses the fact that insulin has a strong/high association with hypertension, insulin is not used for hypertension (that is, contrary to the fact). However, because of a high comorbidity in the diagnosis of diabetes and hypertension, a false association is formed. False associations may also occur between a cardiovascular disease (for example, use of anticoagulants, lower heart rate, strengthened myocardium) and hypertension, and diabetes (for example, use of hypoglycemic medicines and insulin) and hyperlipidemia, etc.

Based on the shortcomings of the conventional art and for the purpose of meeting clinical needs, the processor 130 maintains a first association between each medicine and a disease with the highest association coefficient, and disconnects a second association between each medicine and a disease without the highest association coefficient (step S230). Specifically, each medicine and each disease has a corresponding association coefficient, and a combination is formed accordingly. Each combination includes a medicine, a disease, and a corresponding association coefficient. The processor 130 may further classify these combinations.

In an embodiment, the processor 130 classifies those medicines and one or more diseases with the highest association coefficients corresponding to those medicines into a strong/high association combination, and classifies those medicines and one or more diseases without the highest association coefficients corresponding to those medicines into a weak/low association combination. In other words, for any medicine, the processor 130 determines which of the diseases has the highest association coefficient, and classifies the combination of the medicine and the disease with the highest association coefficient into the strong association combination. On the other hand, the processor 130 classifies other combinations of the same medicine and diseases without the highest association coefficient into the weak association combination.

In addition, the processor 130 maintains the first association of those combinations in the strong association combination, and disconnects the second association of those combinations in the weak association combination. That is, the first association of a disease and a medicine in any combination in the strong association combination is maintained (that is, the first association maintains “yes”), but the association of a disease and a medicine in any combination in the weak association combination is removed (that is, the association changes from “yes” to “no”).

For example, Table (2) shows the combinations to be evaluated:

TABLE 2

| Disease | Medicine |
|---|---|
| Disease 1 | Medicine 1 |
| Disease 2 | Medicine 2 |
| | Medicine 3 |

Before calculating the association coefficient through the evaluating model, the processor 130 establishes the associations (that is, the associations are all “Yes”) of each combination:

TABLE 3

| Disease | Medicine | Association |
|---|---|---|
| Disease 1 | Medicine 1 | Yes |
| Disease 1 | Medicine 2 | Yes |
| Disease 1 | Medicine 3 | Yes |
| Disease 2 | Medicine 1 | Yes |
| Disease 2 | Medicine 2 | Yes |
| Disease 2 | Medicine 3 | Yes |

For example, the association between Disease 1 and Medicine 1 is “Yes” (that is, an association is present), and the rest can be deduced by analogy.

The processor 130 calculates association coefficients Q11, Q12, Q13, Q21, Q22, and Q23 (taking the Q coefficients as an example) through the evaluating model to obtain Table (4):

TABLE 4

| Disease | Medicine | Q coefficient |
|---|---|---|
| Disease 1 | Medicine 1 | Q11 |
| Disease 1 | Medicine 2 | Q12 |
| Disease 1 | Medicine 3 | Q13 |
| Disease 2 | Medicine 1 | Q21 |
| Disease 2 | Medicine 2 | Q22 |
| Disease 2 | Medicine 3 | Q23 |

The processor 130 obtains the strong association combination according to the following functions (1) to (3):

Max(Disease i Medicine 1 Q) (1)

Max(Disease i Medicine 2 Q) (2)

Max(Disease i Medicine 3 Q) (3)

Max( ) refers to the highest association coefficient, i is 1 to 2, and Q is Q11, Q12, . . . , or Q23.

Assuming that the strong association combination is: Disease 1 and Medicine 1, Disease 1 and Medicine 2, and Disease 2 and Medicine 3; the rest are weak association combinations. The processor 130 maintains the association (that is, the aforementioned first association) of these strong associations, and disconnects the association (that is, the aforementioned second association) of those weak associations:

TABLE 5

| Disease | Medicine | Association |
|---|---|---|
| Disease 1 | Medicine 1 | Yes |
| Disease 1 | Medicine 2 | Yes |
| Disease 1 | Medicine 3 | No |
| Disease 2 | Medicine 1 | No |
| Disease 2 | Medicine 2 | No |
| Disease 2 | Medicine 3 | Yes |

For example, the association of Disease 3 and Medicine 1 is “No” (that is, no association), and the rest can be deduced by analogy.

The processor 130 modifies the association coefficients between those diseases and those medicines through the evaluating model according to the first association that is maintained and the second association that is disconnected (step S250). Specifically, unlike step S210, in which an association is established for all combinations (that is, the association of all combinations is “Yes”), some combinations here (for example, the weak association combination) do not have an association (that is, the association is “No”). Therefore, the association coefficients between those diseases and those medicines obtained based on the modification to the associations by the evaluating model should be different from the association coefficients obtained in step S210. In an embodiment, the processor 130 increases the association coefficients in the strong association combination according to the association that is modified. For example, the association coefficients are increased by 20%. In another embodiment, the processor 130 reduces the association coefficients in the weak association combination according to the associations that are modified. For example, the association coefficients are reduced by 50%. In another embodiment, the processor 130 increases the difference between the strong association combination and the weak association combination according to the associations that are modified.

Taking Table (1) as an example, assuming that type 2 diabetes mellitus and metformin, and hypertension and captopril are strong association combinations, and type 2 diabetes mellitus and captopril, and hypertension and metformin are weak association combinations, Table (6) is an example to illustrate the association coefficients that are modified:

TABLE 6

| | | Association coefficient |
|---|---|---|
| | | (taking the Q coefficient |
| Disease | Medicine | as an example) |
| Type 2 diabetes mellitus | Metformin | 17.25081 |
| | Captopril | 0.432935 |
| Hypertension | Metformin | 0.262834 |
| | Captopril | 3.711925 |

In this way, the difference between the strong association combination and the weak association combination may be increased. In addition, the association coefficients of the weak association combination are even close to zero, and these weak association items (for example, common medicines for symptom treatment such as analgesic and anti-inflammatory medicines, first-line antibiotics, etc.) that are not clinically related are excluded.

In addition to the combinations of medicines and diseases, other association variables may be further evaluated. An association variable includes one or more diseases, one or more patient characteristics, and/or one or more visit categories. In an embodiment, the processor 130 may determine the association coefficients between those association variables and multiple medicines through the evaluating model. Similarly, the evaluating model has learned the association coefficients between the association variables and the medicines based on the machine learning algorithm. For example, actual prescriptions and medical record data are used as training samples to understand the degree of association between these variables. Similarly, for any medicine, the processor 130 determines which one of the association variables has the highest association coefficient, and classifies the combination of the medicine and the association variable with the highest association coefficient into the strong association combination. On the other hand, the processor 130 classifies the combination of the same medicine and the association variable without the highest association coefficient into the weak association combination. After obtaining the strong association combination and the weak association combination, the processor 13 may modify the association coefficients between those association variables and those medicines through the evaluating model according to the first association (corresponding to the strong association combination) that is maintained and the second association (corresponding to the weak association combination) that is disconnected. In this way, the association coefficients of the weak association combination may be reduced.

For example, Table (7) illustrates the correspondence between gender, age, disease, medicine and association coefficient:

TABLE 7

| | | | | Association |
|---|---|---|---|---|
| Gender | Age | Disease | Medicine | coefficient |
| Male | 0-19 | Rosacea | Inderal | 24.121 |
| Male | 20-39 | Rosacea | Inderal | 43.182 |
| Male | 40-59 | Rosacea | Inderal | 27.46 |
| Male | 60+ | Rosacea | Inderal | 28.853 |
| Female | 0-19 | Rosacea | Inderal | 10.025 |
| Female | 20-39 | Rosacea | Inderal | 15.109 |
| Female | 40-59 | Rosacea | Inderal | 20.391 |
| Female | 60+ | Rosacea | Inderal | 57.894 |

It is to be noted that Inderal is a very common medicine. If the department is not used as the association variable, Inderal, which is a key medicine that the dermatology department uses in an off-label way for rosacea, is not a reasonable medicine, regardless of age or gender. However, as long as the department is highlighted, it is reasonable for both males and females or any age group to use.

Table (8) is an example to illustrate the correspondence (considering dermatology) between gender, age, disease, medicine, and the association coefficient after modification:

TABLE 8

| | | | | Association |
|---|---|---|---|---|
| Gender | Age | Disease | Medicine | coefficient |
| Male | 0-19 | Rosacea | Inderal | 0.79 |
| Male | 20-39 | Rosacea | Inderal | 0.16 |
| Male | 40-59 | Rosacea | Inderal | 0.159 |
| Male | 60+ | Rosacea | Inderal | 0.198 |
| Female | 0-19 | Rosacea | Inderal | 0.713 |
| Female | 20-39 | Rosacea | Inderal | 0.246 |
| Female | 40-59 | Rosacea | Inderal | 0.484 |
| Female | 60+ | Rosacea | Inderal | 0.56 |

It can be seen that after the introduction of dermatology, all association coefficients are changed to close to zero.

In another case, Etumine, which is used for dementia with behavioral symptoms to control agitation, aggression, or hyperactivity, is mainly prescribed for the elderly regardless of department and gender. If the medicine for dementia is used for a patient under the age of 60, the medicine should be considered an inappropriate medicine regardless of the patient's characteristics. The embodiment of the disclosure may verify that the big data only include prescriptions for males and females over 60 years old. For example, Table (9) is an example to illustrate the correspondence between gender, age, disease, medicine, and the association coefficients after modification:

TABLE 9

| | | | | Association |
|---|---|---|---|---|
| Gender | Age | Disease | Medicine | coefficient |
| Male | 60+ | Dementia | Etumine | 1.758 |
| Female | 60+ | Dementia | Etumine | 1.855 |

In another case, a patient suffered from diabetes and hypertension. The patient used a hypoglycemic agent, metformin, a diuretic, Hydrochlorothiazide, and a calcium channel blocker, Nifedipine in the early stages of diabetes and hypertension, and used insulin and β1-selective adrenergic receptor blocker, Bisoprolol in the late stage. After all the aforementioned prescriptions are entered, the embodiment of the disclosure does not mistakenly associate any blood pressure lowering medicines with diabetes. On the contrary, the embodiment of the disclosure does not associate a hypoglycemic agent or insulin with hypertension. In addition, the embodiment of the disclosure may automatically classify a certain stage of blood pressure lowering medicines to be associated with hypertension, and another stage of blood sugar control medicines to be associated with diabetes.

In summary, in the clinical association evaluating apparatus and the clinical association evaluating method of the disclosure, the associations in the combinations with lower association coefficients are disconnected to change the association coefficients of different combinations. The embodiment of the disclosure may be applied to a variety of evaluating models, remove false associations, and be widely used in actual cases (for example, automatic classification of complex clinical data).

Although the disclosure has been disclosed in the above by way of embodiments, the embodiments are not intended to limit the disclosure. Those with ordinary knowledge in the technical field can make various changes and modifications without departing from the spirit and scope of the disclosure. Therefore, the protection scope of the disclosure is subject to the scope of the appended claims.

What is claimed is:

1. A clinical association evaluating method, comprising:
determining association coefficients between a plurality of diseases and a plurality of medicines through an evaluating model, wherein the evaluating model is trained through a machine learning algorithm;
maintaining a first association between each of the medicines and one of the diseases with the highest association coefficient, and disconnecting a second association between each of the medicines and at least one of the diseases without the highest association coefficient; and
modifying the association coefficients between the diseases and the medicines through the evaluating model according to the first association that is maintained and the second association that is disconnected.

2. The clinical association evaluating method according to claim 1, further comprising:
classifying the medicines and at least one of the diseases with the highest corresponding association coefficient into a strong association combination; and
classifying the medicines and at least one disease without the highest corresponding association coefficient into a weak association combination.

3. The clinical association evaluating method according to claim 2, wherein modifying the association coefficients between the diseases and the medicines through the evaluating model comprises:
increasing the association coefficients in the strong association combination.

4. The clinical association evaluating method according to claim 2, wherein modifying the association coefficients between the diseases and the medicines through the evaluating model comprises:
reducing the association coefficients in the weak association combination.

5. The clinical association evaluating method according to claim 1, further comprising:
determining, through the evaluating model, association coefficients between a plurality of association variables and a plurality of medicines, wherein the association variables comprise at least one of the diseases, a plurality of patient characteristics, and a plurality of visit categories; and
modifying the association coefficients between the association variables and the medicines through the evaluating model according to the first association that is maintained and the second association that is disconnected.

6. A clinical association evaluating apparatus, comprising:
a storage device, adapted for storing a code; and
a processor, coupled to the storage device, configured to load and execute the code so as to execute:
determining association coefficients between a plurality of diseases and a plurality of medicines through an evaluating model, wherein the evaluating model is trained through a machine learning algorithm;
maintaining a first association between each of the medicines and one of the diseases with the highest association coefficient, and disconnecting a second association between each of the medicines and at least one of the diseases without the highest association coefficient; and
modifying the association coefficients between the diseases and the medicines through the evaluating model according to the first association that is maintained and the second association that is disconnected.

7. The clinical association evaluating apparatus according to claim 6, wherein the processor is further configured to:
classify the medicines and at least one of the diseases with the highest corresponding association coefficient into a strong association combination; and
classify the medicines and at least one disease without the highest corresponding association coefficient into a weak association combination.

8. The clinical association evaluating apparatus according to claim 7, wherein the processor is further configured to:
increase the association coefficients in the strong association combination.

9. The clinical association evaluating apparatus according to claim 7, wherein the processor is further configured to:
reduce the association coefficients in the weak association combination.

10. The clinical association evaluating apparatus according to claim 6, wherein the processor is further configured to:
determine, through the evaluating model, association coefficients between a plurality of association variables and a plurality of medicines, wherein the association variables comprise at least one of the diseases, a plurality of patient characteristics, and a plurality of visit categories; and
according to the first association that is maintained and the second association that is disconnected, modify the association coefficients between the association variables and the medicines through the evaluating model.
